(19) United States
(12) Patent Application Publication
(10) Pub. No.: US 2005/0272100 A1
(43) Pub. Date: Dec. 8, 2005

(54) Separation and quantification of biotinylated macromolecules

(76) Inventor: Arthur Karlin, Chappaqua, NY (US)

Correspondence Address: LESLIE GLADSTONE RESTAINO;BROWN RAYSMAN MILLSTEIN & STEINER LLP, 163 MADISON AVENUE, PO BOX 1989, MORRISTOWN, NJ, 07962-1989, US

(21) Appl. No.: 10/860,182

(22) Filed: Jun. 2, 2004

(51) Int. Cl. G01N 33/53; C12Q 1/68

(52) U.S. Cl. 435/7.5

(57) ABSTRACT

The present invention provides a biotin-binding membrane comprising covalently-linked biotin-binding agents, such as avidin and streptavidin, and a kit comprising the membrane. Also provided are methods for the detection and quantification of biotinylated molecules.

### FIELD OF THE INVENTION

The invention disclosed herein generally relates to materials and methods for the separation and quantification of biotinylated molecules.

### BACKGROUND OF THE INVENTION

Biotin, a member of the vitamin B family, is an essential cofactor for the activity of many enzyme systems and found in large quantities in liver, egg yolk, milk, and yeast. It has been extensively used for labeling biological molecules, such as proteins, and more specifically cell surface proteins on intact cells and permeabilized cells (Meier, et al., Immunodetection of biotinylated lymphocyte-surface proteins by enhanced chemiluminescence: a nonradioactive method for cell-surface protein analysis. Anal. Biochem. 204:220-6, 1992; Nesbitt and Horton, A nonradioactive biochemical characterization of membrane proteins using enhanced chemiluminescence. Anal. Biochem. 206:267-72, 1992; Altin, et al., Evidence for an association of CD45 with 32,000-33,000 MW phosphoproteins on murine T and B lymphocytes. Immunol. 83:420-9, 1994; and Altin and Paglet, A one-step procedure for biotinylation and chemical cross-linking of lymphocyte surface and intracellular membrane-associated molecules. Anal. Biochem. 224:382-9, 1995). Biotin derivatives can be prepared by modifying the pentanoic acid carboxyl group without significantly altering the target's physical and biological characteristics. This allows biotin to be conjugated to a number of target molecules with no loss in biological activity, such as the activity to bind avidin and streptavidin.

Streptavidin and avidin have been broadly used in biological and medical science due in large part to their ability to bind biotin specifically. The streptavidin/avidinbiotin interaction has a large number of applications, particularly in the diagnosis and purification areas. In general, a target molecule to be purified or detected is linked or bound either directly to biotin or to a biotinylated intermediate. The biotinylated molecule is then recognized by streptavidin or avidin, which may be linked or bound to a reporter means (such as a peroxidase) or a purification means (such as a resin).

Substances, such as filters, membranes, and beads, bearing streptavidin/avidin have been used for the detection and quantification of biotinylated peptides. For example, U.S. Pat. No. 6,022,951 discloses the covalent linking of streptavidin or avidin to solid supports such as beads, tubes, and membranes. U.S. Pat. Nos. 6,066,462 and 6,348,310 disclose a biotin-binding membrane, which is linked to avidin or streptavidin for the purpose of capturing biotinylated peptides by directly spotting reaction on the membrane. The membrane disclosed in theses patents has been commercialized as the SAM² Biotin Capture Membrane by Promega Corporation, the assignee of the patents. However, no evidence shows that the membrane is suitable for applications other than direct dot-spotting, such as applications requiring the electrical transfer of the biotinylated molecules from a separation gel to a biotin-binding membrane (e.g., Western blot).

### SUMMARY OF THE INVENTION

The present invention provides a biotin-binding membrane comprising covalently-linked biotin-binding agents. In a preferred embodiment, the biotin-binding membrane is a hydrophilic, porous, polymeric membrane with covalently-linked biotin-binding agents, which preferentially captures biotin-tagged proteins and binds untagged proteins poorly. Transfer of proteins from a separation means (e.g., a separation gel) to the biotin-binding membrane can be driven electrically or by pressure, capillary flow, or diffusion. In one embodiment, the biotin-binding agent is avidin or streptavidin. Also provided is a kit for detecting or quantifying biotinylated molecules in a sample comprising the membrane.

The present invention also provides a method for detecting or quantifying biotinylated molecules in a sample, comprising the separation of the biotinylated molecules in a sample according to their physical and/or chemical properties, the electrophoretic transfer of the separated molecules from the gel to a biotin-binding membrane, and the detection and quantification of biotinylated molecules. In one embodiment, the biotin-binding agent is avidin or streptavidin. In another embodiment, the electrophoretic transfer is facilitated using a buffered transfer solution, preferably containing 0.1% (w/v) sodium dodecyl sulphate (SDS), which suppresses non-specific hydrophobic interactions of untagged components with the biotin-binding membrane and permits specific binding of biotin-tagged components.

Additional aspects of the present invention will be apparent in view of the description that follows.

### BRIEF DESCRIPTION OF THE FIGURES

FIG. 1 sets forth the time-course of the covalent attachment of (A) ³H-tyramine and (B) avidin to aldehyde-derivatized filters. (A) Pall UltraBind™ US450 filter squares (1.4 cm×1.4 cm) were shaken at room temperature with 0.3 ml of a mixture of 1 mM tyramine and 125 nM ³H-tyramine in 100 mM NaCl—50 mM NaPi (pH 7.4) (NaCl/NaPi). At the indicated times, the reaction mixture was diluted with 3 ml NaCl/NaPi and shaken 5 min. The diluted mixture was removed, and the filters were washed twice with 3 ml NaCl/NaPi. Scintillation fluid was added to the washed filters, and the incorporated radioactivity was determined and converted to moles based on ³H-tyramine standards. (B) UltraBind™ filters squares (1 cm×1 cm) were shaken with 0.2 ml of 100 μM avidin in NaCl/NaPi—3 mM NaN₃. At the indicated times, the reaction mixture was diluted with 1.5 ml NaCl/NaPi, and the supernatant was removed. The filters were washed four more times with 1.5 ml NaCl/NaPi. The membranes were shaken for 30 min. at room temperature with 400 μl of a mixture of 10 μM d-biotin and 0.2 μM ¹⁴C-d-biotin. The mixture was diluted with 1.5 ml NaCl/NaPi, shaken for 5 min. and removed. The membranes were again washed four more times with 1.5 ml NaCl/NaPi. The incorporated radioactivity was determined by liquid scintillation counting. The points are means of duplicates. The error bars are smaller than the symbols. The curves are least-squares, single-exponential fits.

FIG. 2 shows the result of a Western blot on an avidin coated membrane (ACME) filter and on a nitrocellulose filter of biotinylated and non-biotinylated IgG and reduced IgG. The disulfide bonds in biotinylated and non-biotinylated IgG were either reduced or not. Samples were electrophoresed on a 4-12% Invitrogen NuPAGE BisTris gel, electrophoretically transferred to ACME filter (right panel) or to nitrocellulose (left panel), and detected as described in Examples. The quantities in femtomoles of the indicated components loaded on each gel lane are given above the blot of the lane. The blots were photographed at the digital camera setting of medium resolution and medium sensitivity (f/0.85), with a 5 min. exposure for the ACME blot and a 2 min. exposure for the nitrocellulose blot. The molecular masses in kDa of the molecular weight standards are shown at left.

FIG. 3 depicts the result of Western blot on an ACME filter and on a nitrocellulose filter of biotinylated and non-biotinylated ACh receptor αFLAG subunit and αFLAG-βHA fusion protein. ACh receptor was extracted in SDS/urea from a total membrane fraction of surface-biotinylated TSA-201 cells or from non-biotinylated cells. The extracted proteins were reduced in 10 mM dithiothreitol for 20 min. at 50° C., alkylated with 25 mM N-ethylmaleimide and electrophoresed on a 4-12% NuPAGE BisTris gel. Biotinylated and non-biotinylated α-β fusion protein was diluted and loaded on the gel in SDS/urea. Transfer to ACME and to nitrocellulose and detection of the bands is described in the Examples. The quantities in femtomoles of the indicated components loaded on each lane are given above the lane. The molecular masses of αFLAG and of its aggregates were determined by comparison with standards. The images were digitally photographed at medium sensitivity—medium resolution, at a lens setting of f/0.85, and for biotinylated and non-biotinylated αFLAG, a 1 min. exposure for the ACME blot and a 15 sec. exposure for nitrocellulose blot, and for biotinylated and non-biotinylated αFLAG-βHA fusion protein, a 10 min. exposure for the ACME blot and a 1 min. exposure for nitrocellulose blot.

### DETAILED DESCRIPTION OF THE INVENTION

The present invention provides a biotin-binding membrane, which is covalently modified with a plurality of biotin-binding agents. As used herein, the term “biotin-binding membrane” refers to a membrane suitable for use in an electrical, pressure-driven, or diffusion transfer process, wherein at least one substance, such as a polypeptide or a polynucleotide, is transferred from one subject, such as a polyacrylamide gel, to another subject, such as a membrane-filter. A typical example of electrical transfer processes is a Western blot. As used herein, the term “blot” also includes various derivatives of that blotting technique. For example, the term “Western blot” includes not only the standard Western blot, but also variants such as far-Western blot, Northwestern blot and Southwestern blot. There are a variety of membranes suitable for use as Western blot membranes known in the art including, without limitation, polyvinylidene difluoride (PVDF) membrane, nitrocellulose membrane and nylon membrane. Typically, Western blot membranes are hydrophobic, and the capture of polypeptides or polynucleotides is by non-specific hydrophobic interaction with proteins in general, which results in higher background noise. In a preferred embodiment, the membrane of the invention is a hydrophilic membrane and the transfer takes place in a solution that suppresses hydrophobic interaction and, therefore, enhances the specificity for biotin-tagged components and reduces background noise. The membrane may be any biotin-binding membrane capable of being covalently modified by biotin-binding agents, such as avidin or streptavidin. In one embodiment, the biotin-binding membrane is a modified polyethersulfone (PES) membrane. Another suitable hydrophilic membrane would be composed of cellulose. The membrane may be derivatized, such as by creating aldehyde groups, to facilitate the covalent attachment of biotin-binding agents to the membrane.

The biotin-binding membrane of the present invention is covalently modified with a plurality of biotin-binding agents. As used herein, an “agent” shall include a protein, polypeptide, peptide, nucleic acid (including DNA and RNA), antibody (monoclonal and polyclonal), Fab fragment, F(ab′)₂ fragment, molecule, compound and any combinations thereof. A Fab fragment is a univalent antigen-binding fragment of an antibody and an F(ab′)² fragment is a divalent antigen-binding fragment of an antibody. Unless otherwise indicated, “polypeptide” or “protein” shall include a protein, protein domain, polypeptide or peptide and any fragment or variant or derivative thereof having polypeptide function. The variants preferably have greater than about 75% homology with the naturally-occurring polypeptide sequence, more preferably have greater than about 80% homology, even more preferably have greater than about 85% homology and, most preferably, have greater than about 90% homology with the polypeptide sequence. In some embodiments, the homology may be as high as about 95%, 98% or 99%. These variants may be substitutional, insertional or deletional variants. The variants may also be chemically-modified derivatives: polypeptides which have been subjected to chemical modification, but which retain the biological characteristics of the naturally-occurring polypeptide. In one embodiment, the biotin-binding agents of the present invention may be avidin or streptavidin. In a preferred embodiment, about 0.5 nmol avidin or streptavidin is covalently linked to per cm² of membrane. Biotin-binding agents of the same type or different types may be used to modify a biotin-binding membrane.

Biotin-binding agents may be covalently linked to a membrane directly or through a linker. In one embodiment, the biotin-binding agents, such as avidin or streptavidin, are directly linked to a membrane. For example, the lysyl residues of avidin can form Schiff bases with the aldehyde groups of a chemically modified (PES) membrane. The Schiff base linkages between avidin and the membrane aldehyde groups, which can slowly reverse, may be stabilized, such as by using NaBH₄, to form irreversible secondary amine bonds. In another embodiment, the biotin-binding agents are indirectly linked to a membrane through a linker, such as a C6-C8 linker. Other linkers would be apparent to one skilled in the art.

The present invention further provides a kit for detecting or quantifying biotinylated molecules in a sample, which comprises a biotin-binding membrane covalently modified by a plurality of biotin-binding agents and instructions for use. Other agents, such as antibodies and detecting or reporting agents, may also be included.

The present invention also provides a method for detecting or quantifying biotinylated molecules in a sample, comprising separating or partially separating the biotinylated molecules from other molecules in the sample; electrically transferring the biotinylated molecules to a membrane which is covalently modified with biotin-binding agents; and detecting the presence of or quantifying the biotinylated molecules. The membrane may be any membrane suitable for use in an electrical transfer or pressure or diffusive process including, without limitation, the hydrophilic PES membrane and cellulose membrane and even hydrophobic membranes, such as PVDF membrane, nitrocellulose membrane and nylon membrane. In one embodiment, the biotin-binding agents of the present invention may be avidin or streptavidin. In a preferred embodiment, about 0.5 nmol avidin or streptavidin is covalently linked to per cm² of membrane. Biotin-binding agents of the same type or different types may be used to modify a biotin-binding membrane. Furthermore, biotin-binding agents may be covalently linked to a membrane directly or through a linker.

In one embodiment, samples containing the biotinylated molecules of interest may be subject to at least one separation process before the transfer step. The separation process may be based on any physical or chemical properties of the molecules in the sample, such as molecular weight, shape, polarity, charge or affinity to other molecules. If the biotinylated molecules of interest are biotinylated polypeptides, such as biotinylated cell membrane proteins, gel electrophoresis may be used to resolve the biotinylated polypeptides from other polypeptides in the sample. Depending on the purpose of the application, non-denaturing or denaturing gel electrophoresis may be employed. Optionally, the sample may be subject to a number of different gel electrophoresis processes to improve the resolution of the separation process, such as an isoelectric focusing polyacrylamide gel electrophoresis (PAGE) followed by an SDS-PAGE.

Biotinylated molecules may be electrically transferred from one object to a membrane. For example, biotinylated polypeptides may be transferred from a polyacrylamide gel to a biotin-binding membrane. In one embodiment, the electrical transfer is performed in an aqueous transfer solution, such as a buffer solution. Various agents may be added to the transfer buffer to facilitate the transfer process, for example, detergent may be added to the transfer buffer to facilitate the transfer process by keeping the proteins or other transferred components in solution and by suppressing hydrophobic interactions with the membrane. In one embodiment, the detergent is SDS. The adding of SDS does not significantly interfere with the transfer process when the membrane of the present invention is used. For example, the present invention demonstrated that 85% of biotin is captured by the avidin-coated membrane even in buffer containing SDS in a concentration as high as 0.1% (w/v).

### EXAMPLES

The following examples illustrate the present invention, which are set forth to aid in the understanding of the invention and should not be construed to limit in any way the scope of the invention as defined in the claims which follow thereafter.

### Example 1

### Materials

Avidin and mouse IgG were from Rockland Immunochemicals (Gilbertsville, Pa.). Aldehyde-derivatized, polyethersulfone membrane filters (UltraBind™ US450) were from Pall Corp. (Port Washington, N.Y.). ³H-Tyramine (³H-4-(2-aminoethyl)phenol, 40 Ci/mmol) was from American Radiolabeled Chemicals, Inc (St. Louis, Mo.). ¹⁴C-d-biotin (54 Ci/mol) was from Amersham Pharmacia Biotech (Piscataway, N.J.). Mouse M2 anti-FLAG® IgG, mouse anti-hemagglutinin (HA) IgG, N-biotinyl, N′-[6-maleimidohexanoyl]-hydrazide (biotin maleimide) and diisopropyl fluorophosphate (DFP) were from Sigma-Aldrich Co. (St. Louis, Mo.). NuPAGE NOVEX BisTris gels were from Invitrogen (Carlsbad, Calif.). Horseradish-peroxidase-tagged goat-anti-mouse IgG and Cruz molecular weight standards were from Santa Cruz Biotechnology, Inc. (Santa Cruz, Calif.). Luminescence reagent SuperSignal® West Femto Maximum Sensitivity Substrate, Ultralink Immobilized NeutrAvidin Plus (UINAP) and sulfosuccinimidyl-6-(biotinamido) hexanoate (sulfo-NHS-LC-biotin) were from Pierce Biotechnology Corp. (Rockford, Ill.).

### Example 2

### ACH-Receptor

Modified structural genes for mouse acetylcholine receptor subunits α (αFLAG) and β(βHA) and wild-type genes for γ and δ, were each ligated into the expression vector PCIneo (Promega Corp., Madison, Wis.), as described previously (Wilson and Karlin. The location of the gate in the acetylcholine receptor channel. Neuron 20:1269-81, 1998). In the construct αFLAG, α subunit was altered to contain the octapeptide FLAG-epitope in place of residues 347-354 in the cytoplasmic loop between M3 and M4. In addition, Cys222 was mutated to Ser, Cys418 was mutated to Ala and Val413 was mutated to Ala. In the βHA construct, β was altered to contain the nonapeptide hemagglutinin epitope in place of residues 355-363, and Cys233 was mutated to Ser (βHA). Thus, in both αFLAG and βHA, cysteines that did not normally form disulfides were deleted.

TSA-201 cells (a gift from William Green, University of Chicago) were transfected with plasmids coding αFLAG, βHA, γ, and δ, in the ratio 2:1:1:1 by the calcium-phosphate method, as described before (Wilson and Karlin. The location of the gate in the acetylcholine receptor channel. Neuron 20:1269-81, 1998). This combination was our pseudo-wild type (pWT), in which all other mutations were made. It was expressed on the cell surface as functional receptor.

### Example 3

### Cell-Surface-Biotinylation

Three days after transfection, TSA-201 cells in each 60-mm plate were collected in 2×2 ml of 5 mM EDTA/PBS (PBS: 154 mM NaCl—2.7 mM KCl—9 mM Na₂HPO₄—2 mM NaH₂PO₄—pH 7.4), sedimented at ˜580×g for 3 min. The cells were twice gently suspended in 3 ml 5 mM glucose/high-K buffer (high-K buffer: 140 mM KCl—5 mM NaCl—1.7 mM MgCl₂—1.8 mM CaCl₂—1 mM NaPi—25 mM HEPES—pH 7.4) and sedimented. The cells were suspended in 1 ml of glucose/high-K buffer. Subsequently, 1 ml of 2 mM sulfo-NHS-LC-biotin in high-K buffer was added to the sell suspension. The mixture was slowly mutated for 10 min. at room temperature. To stop the reaction, 1 ml of 15 mM glycine methyl ester/high K buffer was added, and the suspension was mixed for 10 min. The cells were washed twice with 5 ml high-K buffer.

### Example 4

### Total Membrane Fraction

The washed cells from each 60-mm plate expressing ACh receptor, either biotinylated or not, were suspended in 1.2 ml ice-cold DFP/phosphate/EDTA (100 μM di-isopropylfluorophophate (a serine protease inhibitor)—10 mM NaPi (pH 7.0)—1 mM EDTA) and transferred to a small all-glass hand homogenizer (Kimble/Kontes, Vineland, N.J.). The cells were broken with 20 strokes of the pestle, and the suspension was centrifuged in an Airfuge (Beckman Coulter Corp, Fullerton, Calif.) at 29 psi (˜160,000×g) for 3 minutes. The membrane pellets were suspended in 1 ml of ice-cold PE/DFP and sheared by passage 20 times through a 1.5-inch, 22-gauge syringe needle. The membrane was stored in liquid nitrogen.

### Example 5

### Receptor Concentration

The concentration of all ACh-binding sites and, hence, the concentration of αFLAG subunits are determined by a filtration assay for the binding of 125I-α-bungarotoxin (Martin, et al., The contributions of aspartyl residues in the acetylcholine receptor gamma and delta subunits to the binding of agonists and competitive antagonists. J. Biol. Chem. 271:13497-503, 1996). The concentration of α subunit that is biotinylated and, hence, is in cell-surface membrane is determined by the binding of ¹²⁵I-α-bungarotoxin to sites that bind to UINAP NeutrAvidin beads. About 40% of the total α-bungarotoxin-binding sites are bound to UINAP beads and, therefore, about 40% of all αFLAG subunits in the cells were biotinylated.

### Example 6

### αFLAG-βHA Fusion Protein

The genes for αFLAG (with residues 432-449 deleted) and βHA were ligated and inserted into the E. coli expression vector, pQE82L (Qiagen, Inc., Stanford, Calif.). The open reading frame coded for MRGSHHHHHHGIL-αFLAG (432-449 deleted)-EL-βHA-KLN. The molecular weight of this construct is 105,124 Da. The protein was expressed in XL1-Blue strain (Stratagene, La Jolla, Calif.), isolated in inclusion bodies and solubilized in SDS. After reduction of the protein with dithiothreitol, a portion was reacted with biotin maleimide and subsequently with ³H-N-ethylmaleimide to yield biotinylated-³H-αFLAG-βHA. Another portion of reduced αFLAG-βHA was reacted only with ³H-N-ethylmaleimide to yield ³H-αFLAG-βHA. The full-length constructs were purified by electrophoresis in SDS on a 7.5% acrylamide gel, staining one lane of the gel, determining the mobility of the 105-kDa band and elution in SDS of corresponding slices of unstained lanes.

### Example 7

### Biotinylated Mouse IgG

900 μl 1.1 mM (990 nmol) sulfo-NHS-LC-biotin in 20 mM NaPi (pH 7.6) was added to 2 mg (13 nmol) mouse IgG in 100 μl 150 mM NaCl—20 mM NaPi (pH 7.2)—3 mM NaN₃. The solution was mixed for 3 min. at room temperature, and the reaction was stopped by the addition of 50 μl 0.5 M Tris (pH 8.0). After 5 min., the protein was precipitated with 9 volumes of acetone, washed with 5 volumes of 70% ethanol and dried in a vacuum desiccator. The biotinylated IgG was dissolved in 1 ml 2% SDS/10 mM Tris (pH 8.0) and stored in aliquots at −20° C. The final concentration by A_{280 nm} was 1.9 mg/ml. Biotinylated and non-biotinylated IgG was reduced in 10 mM dithiothreitol—2% SDS—4 M urea—100 mM Tris—1 mM EDTA (pH 8.0) for 20 min at 50° C. N-ethylmaleimide was added to 25 mM and reacted for 10 min. at room temperature to alkylate all sulfhydryls.

### Example 8

### Acme Filters

The following protocol is for making 3 cm×4 cm pieces of ACME filter, a convenient and economical size; it can be scaled to any size needed.

(1) Cut 6 pieces, 3 cm×4 cm, of Pall UltraBind™ US450 aldehyde-derivatized polyethersulfone porous filter and put each into a 60-mm tissue culture dish;

(2) Add 1.3 ml of 18.4 μM (1.25 mg/ml) avidin in 100 mM NaCl—50 mM NaPi (pH 7.4)—3 mM NaN₃ (NaCl/NaPi/N₃);

(3) Cover plates, seal with parafilm and shake on titer-plate shaker at medium speed overnight (>14 h) at room temperature;

(4) Remove solution, add 2 ml NaCl/NaPi/N₃, shake 5 min. and remove supernatant (‘wash’);

(5) Add 2 ml 40 mM beta-alanine in NaCl/NaPi/N₃ and shake for 15 min. Remove supernatant;

(6) Wash as in (4);

(7) Add 2 ml 80 mM NaBH₄/0.1 M NaHCO₃ (pH 9.2), shake 30 min. Remove supernatant;

(8) Wash as in (4);

(9) Add 2 ml 0.1 M NaPi (pH 8.0), shake 5 min. Remove supernatant;

(10) Add 2 ml 10 mM succinic anhydride/4.8% DMSO/0.1 M NaPi (pH 8.0) (diluted from 210 mM succinic anhydride/DMSO). Shake 60 min. Remove supernatant;

(11) Wash twice as in (4); and

(12) Cover with 100N50P/N₃, seal with parafilm and store at 4° C. Use within 2 weeks.

### Example 9

### Western Blot

SDS-PAGE was run on pre-cast Invitrogen (Carlsbad, Calif.) Novex NuPage 4%-12% Bis-Tris Gels, 1-mm thick, 15 wells, in an Invitrogen Xcell Surelock Mini-Cell chamber. Other gels, such as Pre-cast NuPAGE 10% gels and home-made Laemmli-type gels, are also suitable for the purpose of the present invention. The running buffer was NuPAGE SDS/MOPS (50 mM Tris base—50 mM MOPS—0.1% SDS—1 mM EDTA (pH 7.7). The samples were in 2% SDS/4 M urea/100 mM Tris (pH 8.0)/1 mM EDTA, with Pyronin Y as a tracking dye. No glycerol was necessary when the sample contained 4 M urea (Other sample buffer compositions, including Laemmli sample buffer (Laemmli, U.K. Cleavage of structural proteins during the assembly of the head of bacteriophage T4. Nature 227:680-685, 1970), may also be used.). A 15-μl sample was loaded in each lane. The gels were run at 150 V for ˜40 min. until the dye front had migrated about 4 cm.

Transfer to ACME filter was carried out in a BioRad Mini Trans-blot Cell. The transfer buffer was 0.1% SDS/NuPage transfer buffer (TB). TB contains 25 mM Bicine—25 mM BisTris—1 mM EDTA (pH 7.2). A 3 cm×4 cm ACME filter was laid on the gel. Two filters side by side covered the 15 lanes of the gel. The transfer was run overnight at 20 V at room temperature. Slow transfer to ACME filters without excessive heating gives better selectivity for biotinylated proteins than does rapid transfer. Transfer to nitrocellulose is in 10% methanol/TB (no SDS) and is run overnight at 20 V at ˜5° C.

Each filter was developed in a separate dish. The filter was shaken 1 h in blocking buffer (5% nonfat dry milk powder—1% BSA—0.1% Tween—20 in TBS (137 mM NaCl—20 mM Tris (pH 7.6)), 1 h in M2 mouse anti-FLAG antibody (or other appropriate primary antibody) diluted 1000 times in blocking buffer, 4×15 min in 0.1% Tween/TBS, 1 h in HRP-anti-mouse IgG diluted 3750× in blocking buffer and 9×10 min in 0.1% Tween/TBS. Each 3 cm×4 cm filter was covered with 2 ml of Pierce SuperSignal West Femto Maximum Sensitivity Substrate. After 3 min., each filter was digitally photographed (Alpha Innotech Corp., San Leandro, Calif.). The densities of the bands on the blot images were quantified using Alpha Innotech software.

Discussed below are results obtained by the inventors in connection with the experiments of Examples 1-9.

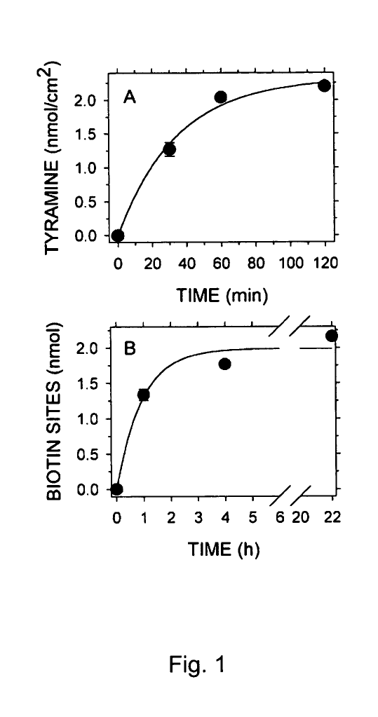

UltraBind US450 polyethersulfone membrane (UB membrane) is thin, porous, hydrophilic and derivatized with aldehyde groups (Pall Lifesciences, Inc.). It reacts with primary amines to form Schiff bases. The rate constant for this reaction and the capacity of the membrane were determined using a radioactive primary amine, ³H-tyramine (FIG. 1A). At pH 7.4 and ˜23° C., the rate constant is 29±5 L/Mol×min (from pseudo-first-order exponential fit of means of duplicates), and the maximum incorporation is 1.8±0.3 nmol per cm².

Avidin, which has 4 biotin binding sites, is a 69,000-Da tetramer of glycosylated subunits. Each subunit has 10 lysyl residues, which can form Schiff bases with aldehyde groups. The rate constant for the covalent attachment of biotin-binding sites to UB membrane at pH 7.4 and room temperature is 180±50 L/(Mol×min) (fit of means of duplicates) (FIG. 1B). The maximum incorporation of active biotin-binding sites was 1.7±0.2 (n=13) nmol per cm².

Because the avidin-biotin complex is known to resist dissociation even under harsh conditions, whether avidin covalently linked to UB membranes would bind biotin in SDS was further tested. UB membranes (1 cm²) were reacted overnight with 70 μM avidin and washed. The binding of 5 μM ¹⁴C-biotin in various concentrations of SDS in 192 mM glycine—25 mM Tris buffer (pH 8.3) was determined as in FIG. 1B. In 0, 0.0125%, 0.025%, 0.05% and 0.1% SDS, the mean binding was 1.65±0.06, 1.70±0.04, 1.69±0.05, 1.51±0.02 and 1.40±0.004 nmol ¹⁴C-biotin/cm² (n=2), respectively. The results indicate that, even in 0.1% SDS, the binding was 85% of that in the absence of SDS.

Following the reaction of UB membrane with avidin, unreacted aldehyde groups were reacted with excess β-alanine, which adds negative charge to the filter. The Schiff base linkages of both avidin and β-alanine to the filter, which can slowly reverse, were reduced with NaBH₄ to form irreversible secondary amines. Finally, the positive charge of the filter, due to the net positive charge of avidin and the protonation of the secondary amine linkages, was partly offset by succinylation of avidin lysines. Succinylation (Finn, et al., Hormone-receptor studies with avidin and biotinylinsulin-avidin complexes. J. Biol. Chem. 255:5742-6, 1980; Holtzman, et al., Electron microscopy of complexes of isolated acetylcholine receptor, biotinyl-toxin, and avidin. Proc. Natl. Acad. Sci. U.S.A. 79:310-4, 1982) and acetylation (Bayer, et al., Sodium dodecyl sulfate-polyacrylamide gel electrophoretic method for assessing the quaternary state and comparative thermostability of avidin and streptavidin. Electrophoresis 17:1319-24, 1996) of avidin, which has 10 lysines and 7 arginines per subunit and a pI of 10, have been used previously to decrease avidin's positive charge and also its nonspecific binding to negatively charged molecules. Neither reaction with β-alanine, reduction with NaBH₄, nor succinylation decreased the biotin-binding of the ACME filters. ACME filters produced according to the present invention were stored in NaCl/NaPi/N₃ at 4° C. and its binding of ¹⁴C-biotin in 0.1% SDS/TB was determined at 1, 2, 6, 8, 15, and 22 days. Over this period of time, there was no significant decrease in biotin binding: the mean of the six determinations was 1.5±0.1 nmol/cm².

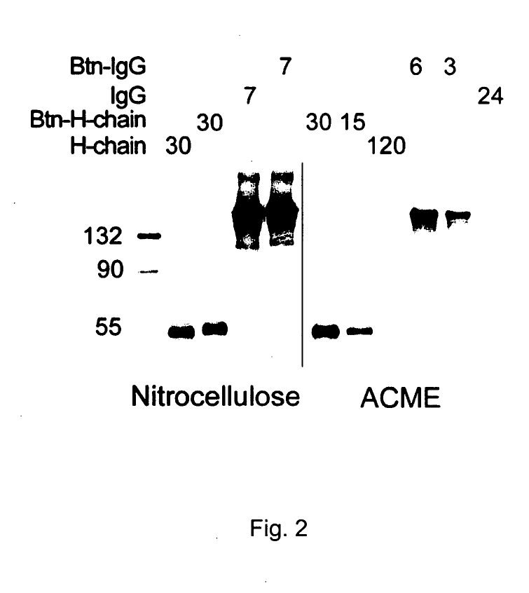

The utility of the ACME filters in Western blotting was first tested with biotinylated and non-biotinylated forms of intact mouse IgG and reduced and dissociated IgG heavy chain (H-chain). These proteins were electrophoresed and transferred to an ACME filter and, for comparison, to a nitrocellulose filter (FIG. 2). Horse-radish-peroxidase (HRP)-labeled goat anti-mouse IgG antibody was used to detect IgG and H-chain. Equal loads of biotinylated and non-biotinylated IgG and biotinylated and non-biotinylated H-chain gave approximately equally dense bands on a nitrocellulose filter. By contrast, 3 fmol of biotinylated IgG was readily detected on an ACME filter, whereas 24 fmol of non-biotinylated IgG was undetectable. Similarly, 15 fmol of biotinylated H-chain was readily detected on an ACME filter, whereas 120 fmol of non-biotinylated H-chain was undetectable. The results indicate that ACME filters specifically capture biotinylated IgG and H-chain. Although femtomoles of biotinylated IgG and biotinylated H-chain are readily detected on ACME filters after a few minutes of exposure, in general a shorter exposure is sufficient to give equally dense bands on a nitrocellulose filter. The ACME filters are clearly compatible with protein-detection by antibody binding and HRP-driven chemoluminescence.

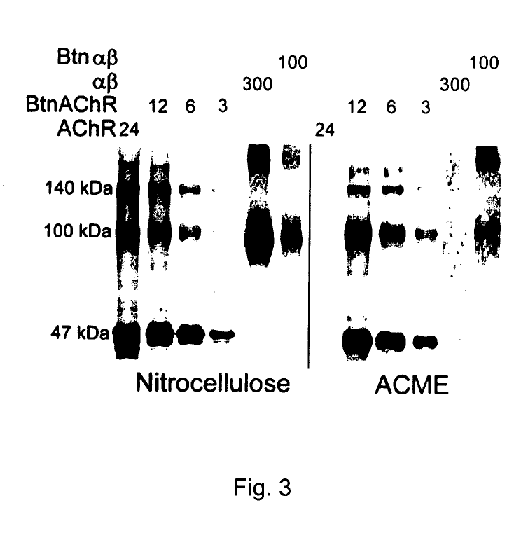

Blotting on ACME and nitrocellulose filters was also tested with biotinylated and non-biotinylated acetylcholine receptor containing αFLAG subunit (˜47 kDa) and with biotinylated and non-biotinylated αFLAG-βHA fusion protein (˜105 kDa) (FIG. 3). These proteins were detected using a mouse anti-FLAG antibody followed by HRP-labeled goat anti-mouse IgG antibody. On ACME filters, 3 femtomoles of αFLAG were readily detected and proportional signals were given by 3, 6 and 12 fmol αFLAG; however, 24 fmol of non-biotinylated αFLAG were undetectable. After a five times longer exposure (5 min.), 24 fmol of non-biotinylated αFLAG gave a light band (not shown). The results indicate that ACME captures biotinylated αFLAG approximately 100 times more efficiently than nonbiotinylated αFLAG. By contrast, both biotinylated and non-biotinylated αFLAG gave signals proportional to their inputs when blotted on nitrocellulose filters. As above, the ACME blots have to be exposed approximately 4 times as long as the nitrocellulose blots to obtain equal dense bands.

Similarly, 100 fmol of biotinylated αFLAG-βHA fusion protein were readily detected on ACME filters, whereas 300 fmol of non-biotinylated αFLAG-βHA fusion protein were not detectable. By contrast, on nitrocellulose filters, these two samples were detected proportionally.

For both water-soluble proteins like IgG and detergent-soluble protein like ACh receptor subunits, ACME blotting is specific for biotinylated proteins compared to non-biotinylated proteins and can be used with chemoluminescence reagents to detect biotinylated proteins at the femtomole level. Biotinylation of membrane proteins with a membrane-impermeant reagent allows the sensitive detection of cell-surface proteins and could be used, for example, to follow their turnover at the cell-surface. ACME blotting could also be used to detect proteins engineered with a biotinylation consensus sequence and biotinylated enzymatically by biotin holoenzyme synthetase either in vitro or in vivo.

The examples clearly demonstrate that the filters provided by the present invention are very sensitive and powerful tools, capable of capturing, detecting and quantifying both water-soluble proteins (e.g., IgG) and hydrophobic proteins (e.g., ACh receptor subunit constructs) in the fmol range.

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A biotin-binding membrane, wherein the membrane is covalently modified with at least one biotin-binding agent.

2. The membrane of claim 1, wherein the membrane is a Western blot membrane.

3. The membrane of claim 1, wherein the membrane is selected from the group consisting of polyethersulfone membrane, cellulose membrane, polyvinylidene difluoride membrane, nitrocellulose membrane and nylon membrane.

4. The membrane of claim 1, wherein the at least one biotin-binding agent is selected from the group consisting of avidin, streptavidin or the variant, derivative or combination thereof.

5. The membrane of claim 4, wherein the binding capacity of the membrane is about 1.7 nmol biotin/cm².

6. A kit for detecting or quantifying biotinylated molecules in a sample, comprising a biotin-binding membrane, wherein the membrane is covalently modified with at least one biotin-binding agent.

7. A method for detecting or quantifying biotinylated molecules in a sample, comprising:
(1) separating, or partially separating, the biotinylated molecules from a plurality of molecules in the sample;
(2) transferring the biotinylated molecules to a membrane, wherein the membrane is covalently modified with at least one biotin-binding agent; and
(3) detecting or quantifying the biotinylated molecules.

8. The method of claim 7, wherein the membrane is a Western blot membrane.

9. The method of claim 7, wherein the membrane is selected from the group consisting of polyethersulfone membrane, cellulose membrane, polyvinylidene difluoride membrane, nitrocellulose membrane and nylon membrane.

10. The method of claim 7, wherein the at least one biotin-binding agent is selected from the group consisting of avidin, streptavidin or the combination thereof.

11. The method of claim 7, wherein the transfer is facilitated using transfer solution containing detergent.

12. The method of claim 11, wherein the detergent is SDS.

13. The method of claim 12, wherein the concentration of SDS is about 0.1% (w/v).

14. The method of claim 7, wherein the biotinylated molecules are separated from other molecules in the sample by gel electrophoresis.

15. The method of claim 14, wherein the gel electrophoresis is polyacrylamide gel electrophoresis.

16. The method of claim 7, wherein the biotinylated molecules are biotinylated polypeptides.

17. The method of claim 7, wherein the transfer is an electrical transfer.
